(19) United States
(12) Patent Application Publication
(10) Pub. No.: US 2025/0057655 A1
(43) Pub. Date: Feb. 20, 2025

(54) SYSTEM AND METHOD FOR IMMUNOMODULATORY ENHANCEMENT OF IMPLANT BONE INTEGRATION

(71) Applicant: Loubert S. Suddaby, Orchard Park, NY (US)

(72) Inventor: Loubert S. Suddaby, Orchard Park, NY (US)

(21) Appl. No.: 18/449,026

(22) Filed: Aug. 14, 2023

(51) Int. Cl. A61F 2/28; A61K 33/06; A61K 38/19

(52) U.S. Cl. CPC A61F 2/28; A61K 33/06; A61K 38/19; A61F 2002/2817

(57) ABSTRACT

An immunomodulatory implant comprising an inner portion including a metal or polymer, an intermediate portion arranged on the inner portion and including a calcium compound, and an outer portion arranged on the intermediate portion, the outer portion including a calcium containing carrier substrate and an immunomodulatory enhancement which favors osteogenic bone growth into the implant such that a stable bone implant integration occurs.

### FIELD

The present disclosure relates to osteoimmunology and bone morphogenesis designed to enhance the integration of implants into the bony skeleton to facilitate the repair or restoration of skeletal functions through targeted surgical intervention.

### BACKGROUND

With an aging population, spine and joint degeneration has become an increasingly problematic affliction of mankind. Degeneration of simple joints such as hips and knees are common as is the degeneration of more complex joints found in the human spine. Fortunately, bony joints can be entirely replaced, including the more complex joints found in the axial skeleton. Appendicular joint replacement surgery enjoys greater success than axial joint replacement surgery, which is still in its infancy.

While the bony skeleton can repair micro stress fractures that occur with daily use, the implants to replace joints cannot, thereby necessitating that the devices used to replace joints or to fuse spinal segments be made sufficiently robust that they can withstand cyclical and episodic traumas the body may encounter over many years.

Consequential to this, joint replacement implants or spinal fusion implants are often made of metal or special synthetic polymers that are strong enough to withstand years of cyclical motion as well as the various traumas expected in the course of a normal life. Because the implants need to be suitably robust, their modulus of elasticity rarely mimics that of normal bone and they are consequently much stiffer than any skeletal substrate. This creates significant and repetitive forces at the bone device interface. Moreover, repetitive movement at the bone device interface frequently leads to loosening and in some cases, subsidence of the device into the softer bone, negating its functional benefit. Loosening or subsidence of implants necessitating revision surgery is far more common than failure of the implant itself, resulting in substantial cost to society as well as patient morbidity since revision surgeries are far more likely to result in surgical complication.

The most important factor in preventing the loosening of implants is osseointegration whereby the surrounding bone grows onto or into the implant and maintains a solid and stable bone/implant interface despite cyclical stress. Motion at this interface frequently pushes the softer bone away from the implant with the resultant space being filled with fibrous tissue which cannot hold the implant securely. A fibrous union thereby permits micro and even macro movement, which compromises device effectiveness. Micromovement alone stimulates mechanoreceptors and nociceptors (or nocireceptors) in bone resulting in persistent pain thereby leading to suboptimal outcomes requiring revision surgery(s).

Another important factor in preventing subsidence or loosening is osteointegration whereby the bone surface contact is maintained to a sufficient degree such that fibrous tissues cannot proliferate within the contact surface area, thereby thwarting micromotion, which eventually contributes to pain and loosening. Osteointegration occurs when the bone grows onto, or preferably into the implant and retains the capacity to remodel such that bony micro stress fractures are repaired as occurs in normal bone.

While other reasons for implant failure can occur (e.g., osteopenia, surgical technique, infections, etc.), most implant loosening is due to failure of osteointegration at the bone implant interface. Indeed, the most common cause for implant failure is aseptic loosening, which is seen on Xray as a radiolucent line or halo around the implant. This results in pain with movement and is the very symptom for which most surgeries are done. Revision of such a complication is technically demanding, frequently associated with surgical complications and generally has a significant reduction in patient satisfaction over the original surgery.

Thus, there is a need for a system and method to mitigate the need for revision surgeries necessitated by implant loosening.

### SUMMARY

The present disclosure is directed to one or more embodiments of an immunomodulatory implant, comprising an inner portion including a metal or polymer, and an outer layer arranged on the inner portion and forming an outer surface, the outer layer including a carrier and an immunomodulatory enhancement.

In an exemplary embodiment, the carrier comprises fibrin. In an exemplary embodiment, the carrier comprises hydrogel. In an exemplary embodiment, the carrier comprises an adhesive. In an exemplary embodiment, the immunomodulatory enhancement comprises an antigen. In an exemplary embodiment, the immunomodulatory enhancement comprises a cytokine. In an exemplary embodiment, the outer layer comprises a plurality of pores, and the carrier and the immunomodulatory enhancement extend radially inward from the outer surface into the plurality of pores.

In an exemplary embodiment, the immunomodulatory implant further comprises an intermediate layer arranged radially between the inner portion and the outer layer. In an exemplary embodiment, the intermediate layer comprises a calcium compound. In an exemplary embodiment, the intermediate layer further comprises an antigen and/or a cytokine. In an exemplary embodiment, the inner portion comprises a porosity and a radial center, and the porosity increases in a radially outward direction from the radial center. In an exemplary embodiment, the carrier comprises a paste or putty derived from xenograft or allograft bone.

The present disclosure is directed to one or more embodiments of an immunomodulatory implant, comprising an innermost layer including a first porosity, the innermost layer forming a radial center, an intermediate layer arranged on the innermost layer, the intermediate layer comprising a second porosity, the second porosity being greater than the first porosity, and an outer layer arranged on the intermediate layer and forming an outer surface, the outer layer including a calcium compound and an immunomodulatory enhancement.

In an exemplary embodiment, the first porosity increases in a radially outward direction from the radial center to the outer surface. In an exemplary embodiment, the second porosity increases in the radially outward direction. In an exemplary embodiment, the innermost layer, the intermediate layer, and the outer layer comprise a metal. In an exemplary embodiment, the outer surface comprises a first plurality of portions comprising a metal or a polymer and a second plurality of portions comprising the calcium compound. In an exemplary embodiment, the second plurality of portions are arranged as islands with respect to the first plurality of portions. In an exemplary embodiment, the immunomodulatory enhancement is arranged only on the second plurality of portions. In an exemplary embodiment, the implant transitions from 100% metal or polymer material at the radial center radially outward to 100% calcium compound and immunomodulatory enhancement at the outer surface in a graduated fashion.

The present disclosure is directed to one or more embodiments of an immunomodulatory implant comprising an inner portion including a metal, polymer, or other biocompatible material designed for use as a bio-mechanical implant, an intermediate portion arranged on the inner portion and including a calcium compound, and an outer portion arranged on the intermediate portion, the outer portion including a carrier and an immunomodulatory enhancement.

The present disclosure is directed to one or more embodiments of a biologically compatible and resorbable adhesive substance into which is admixed one or more antigens having the capability of attracting and inducing M0 macrophages or monocytes and resident macrophages (osteomacs) to a surgical implant by way of an antigen gradient, and/or by the addition of cytokines that selectively favor or bias this process, or other biochemical alteration. It is also desirous that the antigen facilitate the conversion of the recruited M0 macrophage to the M1 and ultimately the M2 macrophage and their subsets. The adhesive substance could be applied to the surface of any implant but preferably a porous implant and one in which a calcium containing compound such as calcium phosphate, calcium carbonate, or hydroxyapatite have been bonded. In an exemplary embodiment, the adhesive substance may comprise one or more of platelet rich plasma cells, fibrin glues, alginate, collagen gels, synthetic polymers, and hydrogels.

In an exemplary embodiment, platelet rich plasma may be made from concentrate of whole blood which has been centrifuged to remove red blood cells. Because platelet rich plasma is derived from blood, it contains growth factors and cytokines which have been shown to encourage tissue healing and may serve as a vessel to carry and apply M0 macrophage stimulating antigen to an implant surface.

In an exemplary embodiment, fibrin glue, which is a biologic adhesive that uses a combination of fibrinogen and thrombin to emulate the final stages of clot formation. The fibrin glue comprises a salutation of concentrated human fibrinogen which is activated by the addition of bovine thrombin and calcium chloride. The resultant amalgam can be impregnated with antigen which then adheres to the implant like a clot and gradually elutes the antigen as the clot dissolves. Any paste (e.g., allograft, bone putty) or flowable material biocompatible and resorbable carrying agent capable of containing antigens that stimulate the innate immune system can be employed.

In an exemplary embodiment, the adhesive is a hydrogel, which can be constructed from absorbable biocompatible polymers and retain a large amount of water which aids in the elution of antigen from the gel to aid providing a chemical gradient along which M0 macrophages can be attracted. The hydrogels can be formulated from synthetic or natural polymers of sugars or proteins to create scaffolds that favor and facilitate cell migration. The hydrogels can also be 3D printed such that a scenario of a 3D printed implant transitioning to a hydroxyapatite surface and then to a hydrogel in a graduated basis is conceived. This includes 3D printing of the antigen containing hydrogel on the implant surface.

The present disclosure is directed to one or more embodiments of an osseoinductive, biointegrating implant including a 3D printed implant of titanium alloy with the implant transitioning from a solid to a porous surface, onto which is printed or otherwise applied, hydroxyapatite, calcium phosphate, and/or calcium carbonate and finally a 3D printed hydrogel into which antigens capable of stimulating the immune system can be imbibed, or even printed into the hydrogel.

The present disclosure is directed to one or more embodiments of an implant including a bioactive additive. In an exemplary embodiment, the implant comprises a metal inner portion (e.g., titanium) and a transitioning outer portion. The transitioning outer portion transitions from 100% metal (e.g., titanium) and 0% calcium compound at its innermost portion (i.e., at the interface with the inner portion) to 100% calcium compound (e.g., calcium phosphate, calcium carbonate, hydroxyapatite, etc.) and 0% metal at its outermost surface. The calcium compound is gradually phased in, for example using 3D printing, such that the calcium compound is not necessarily a coating but an integral part of the implant itself. That is to say that the implant is a calcium metal amalgam (i.e., metal bone) rather than an implant that has been coated with a calcium compound after manufacturing. In an exemplary embodiment, the conversion/transition from metal to calcium compound occurs over a 3-5 mm distance in a radially outward direction to the outer perimeter of the implant.

The present disclosure is directed to one or more embodiments of a porous implant, either coated with one or more calcium products or uncoated, sprayed or covered with antigen and/or cytokine containing glue at or around the time of surgery to facilitate bone apposition and integration.

The present disclosure is directed to one or more embodiments of an immunomodulatory implant comprising a porous titanium inner portion, a first layer comprising a calcium containing compound coating the inner portion, and a second layer comprising a carrier and an antigen(s) and or antigen(s) and selected cytokine(s). In an exemplary embodiment, the first layer is applied to the inner portion via plasma spraying. In an exemplary embodiment, the carrier comprises one of hydrogel, fibrin, and adhesive. In an exemplary embodiment, the second layer is sprayed onto the first layer. In an exemplary embodiment, the inner portion, the first layer, and the second layer are 3D printed.

The present disclosure is directed to one or more embodiments of an immunomodulatory implant designed to enhance the integration of metal or synthetic or semi-synthetic implants or even pure allograft or xenograft implants into the bony skeleton to facilitate the repair or restoration of axial and appendicular skeletal functions through targeted surgical intervention.

These and other objects, features, and advantages of the present disclosure will become readily apparent upon a review of the following detailed description of the disclosure, in view of the drawings and appended claims.

### BRIEF DESCRIPTION OF THE DRAWINGS

The accompanying drawings are incorporated herein as part of the specification. The drawings described herein illustrate embodiments of the presently disclosed subject matter and are illustrative of selected principles and teachings of the present disclosure, in which corresponding reference symbols indicate corresponding parts. However, the drawings do not illustrate all possible implementations of the presently disclosed subject matter and are not intended to limit the scope of the present disclosure in any way.

FIG. 1 is a cross-sectional view of an immunomodulatory implant system.

FIG. 2 is a detail view of the immunomodulatory implant system taken generally along Detail 2 in FIG. 1.

FIG. 3 is a partial cross-sectional view of the immunomodulatory implant shown in FIG. 1, being coated with an immunomodulatory enhancement.

FIG. 4 is a cross-sectional view of an immunomodulatory implant system.

FIG. 5 is a detail view of the immunomodulatory implant system taken generally along Detail 5 in FIG. 4.

FIG. 6 is a detail view of the immunomodulatory implant system taken generally along Detail 6 in FIG. 4.

FIG. 7 is a partial elevational view of the immunomodulatory implant shown in FIG. 4.

### DETAILED DESCRIPTION

At the outset, it should be appreciated that like drawing numbers on different drawing views identify identical, or functionally similar, structural elements. It is to be understood that the claims are not limited to the disclosed aspects.

Furthermore, it is understood that this disclosure is not limited to the particular methodology, materials and modifications described and as such may, of course, vary. It is also understood that the terminology used herein is for the purpose of describing particular aspects only, and is not intended to limit the scope of the claims.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments.

It should be appreciated that the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims. The term “approximately” is intended to mean values within ten percent of the specified value.

“Immunomodulatory implant” as used herein is intended to mean an implant including a substance that actively triggers a response by the body's immune system, so as to facilitate a more optimal bony skeletal repair than that of an implant which does not comprise such immunomodulatory substance.

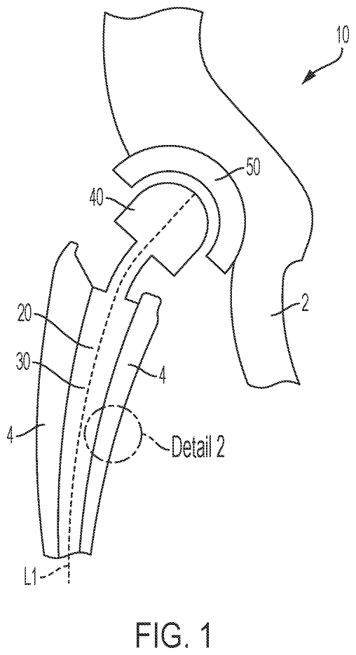

Adverting now to the figures, FIG. 1 is a cross-sectional view of immunomodulatory implant system 10 including immunomodulatory implant 20. In FIG. 1, immunomodulatory implant 20 is embodied as a hip replacement implant shown in anatomic position, namely, within femur bone 4 and pelvic bone 2; although, it should be appreciated that immunomodulatory implant 20 can be embodied as any bone implant and the present disclosure is not to be limited to hip replacement implants.

In an exemplary embodiment, and as shown, immunomodulatory implant 20 comprises femoral stem 30 and femoral head 40. Femoral stem 30 is operatively arranged to engage femur bone 4. For example, femoral stem 30 may be arranged in a longitudinal hole arranged in femur bone 4. Femoral head 40 is arranged to pivotably engage bone 2, in particular, the acetabulum of bone 2. In an exemplary embodiment, immunomodulatory implant 20 further comprises acetabular cup 50 arranged to be secured within bone 2. In such exemplary embodiment, femoral head 40 is arranged to pivotably engage acetabular cup 50.

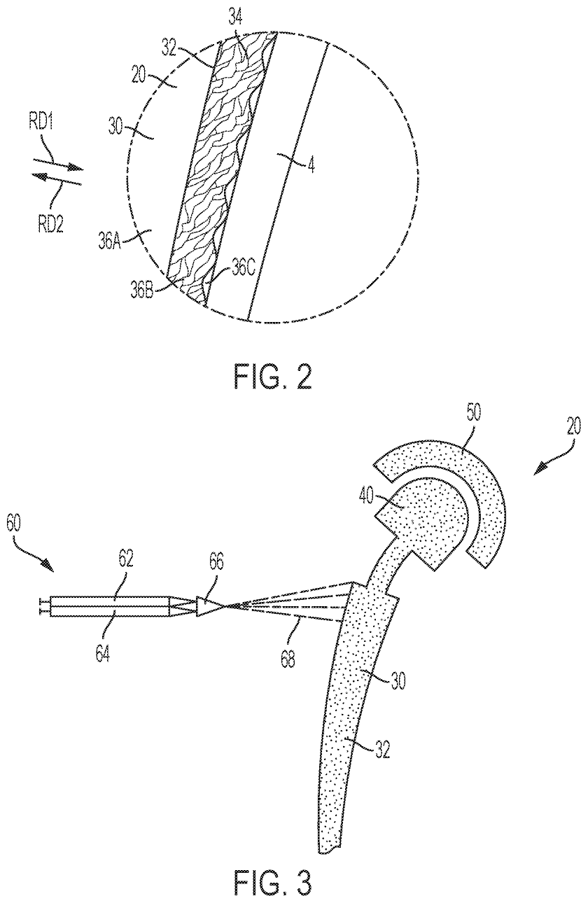

FIG. 2 is a detail view of immunomodulatory implant system 10 taken generally along Detail 2 in FIG. 1. Immunomodulatory implant 20 comprises outer surface 32 and layer 34. It should be appreciated that outer surface 32 denotes the outer surface of femoral stem 30 and femoral head 40. In an exemplary embodiment, outer surface 32 further denotes the outer surface of acetabular cup 50. Layer 34 at least partially covers immunomodulatory implant 20. In some embodiments, layer 34 completely covers immunomodulatory implant 20 (i.e., outer surface 32 is completely coated with layer 34). In an exemplary embodiment, layer 34 comprises at least one of calcium, an antigen, hydrogel, fibrin or fibrin glue, and adhesive.

In an exemplary embodiment, and as best shown in FIG. 2, implant 30 comprises a plurality of portions or layers, for example, portions or layers 36A-36C. In an exemplary embodiment, portion 36A comprises a metal. Portion 36A may comprise a dense metal. In an exemplary embodiment, portion 36A comprises titanium, steel, or cobalt-chrome. In an exemplary embodiment, portion 36A comprises a dense porous or solid metal. In an exemplary embodiment, portion 36A comprises a polymer or plastic, for example, poly ether ether ketone (PEEK). In an exemplary embodiment, portion 36A increases in porosity from its radial center represented by line L1 shown in FIG. 1, in a radially outward direction represented by radial direction RD1 shown in FIG. 2.

In an exemplary embodiment, portion 36B is arranged on portion 36A and comprises a calcium containing compound, for example, calcium phosphate, calcium carbonate, and/or hydroxyapatite. In an exemplary embodiment, portion 36B comprises a calcium containing compound and an antigen (e.g., lipopolysaccharide or lipoteichoic acid) and/or a cytokine (e.g., interferon, interleukin, or growth factors). In an exemplary embodiment, portion 36B is a porous layer that exhibits the porosity and density of trabecular or cancellous bone, which encourages and allows the ingrowth of bone into immunomodulatory implant 20. In an exemplary embodiment, portion 36B is 3D printed onto portion 36A. In an exemplary embodiment, portion 36B is applied to portion 36A via one or more plasma spray coating processes.

In an exemplary embodiment, portion 36C comprises a paste including an antigen, for example, lipopolysaccharide, lipoteichoic acid, and/or a cytokine. The paste may be impregnated or with the antigen and/or cytokine. The paste is applied to porous portion 36B (e.g., hydroxyapatite) to foster bone ingrowth and integration, thereby resulting in better fusion between immunomodulatory implant 20 and surrounding bone, and a lower likelihood of loosening (i.e., of stem 30, head 40, and/or acetabular cup 50).

In an exemplary embodiment, portion 36C comprises hydrogel including an antigen and/or a cytokine. The hydrogel may be impregnated or with the antigen and/or cytokine. In an exemplary embodiment, portion 36C is 3D printed with some portions containing hydrogel and other portions containing the antigen and/or cytokine.

In an exemplary embodiment, portion 36C comprises fibrin glue including an antigen and/or a cytokine. The fibrin glue may be impregnated or coated with the antigen and/or cytokine. In an exemplary embodiment, portion 36C is 3D printed with some portions containing fibrin glue and other portions containing the antigen and/or cytokine.

In an exemplary embodiment, portion 36C comprises adhesive including an antigen and/or a cytokine. The adhesive may be impregnated or coated with the antigen and/or cytokine. In an exemplary embodiment, portion 36C is 3D printed with some portions containing adhesive and other portions containing the antigen and/or cytokine.

In an exemplary embodiment, the implant may comprise a topical antibiotic to enhance intravenous antibiotics given at the time of surgery to aid in the preventions of infections, for example, vancomycin, clindamycin, cephalosporin, etc. (i.e., an immunomodulatory paste containing an antibiotic). In an exemplary embodiment, the antigen and/or cytokine may be coupled with the antibiotic to mitigate surgical post-operative infection.

FIG. 3 is a partial cross-sectional view of immunomodulatory implant 20 shown being coated with an immunomodulatory enhancement (e.g., fibrin, hydrogel, or adhesive including an antigen and/or a cytokine) according to one exemplary embodiment. In such exemplary embodiment, immunomodulatory implant 20 is being sprayed with the immunomodulatory enhancement. For example, to spray the immunomodulatory enhancement onto implant 20, spraying device 60 may be used. Spraying device 60 is a double barreled syringe comprising chamber 62, chamber 64, and nozzle 66. Chamber 62 comprises antigen and/or cytokine and fibrinogen and chamber 64 comprises thrombin. As the plunger(s) is activated, the antigen and/or cytokine and fibrinogen in chamber 62 and the thrombin in chamber 64 are forced into and mixed within nozzle 66. The two components, fibrinogen (e.g., lyophilized pooled human concentrate) and thrombin (e.g., from bovine or human), are mixed immediately before application to form fibrin glue. The mixture of antigen and/or cytokine, fibrinogen, and thrombin is then expelled from nozzle 66 as spray 68 to coat immunomodulatory implant 20. In an exemplary embodiment, spray 68 is applied directly to surface 32 of immunomodulatory implant 20. In such an exemplary embodiment, surface 32 may comprise a high porosity (e.g., greater than 50%), which encourages the ingrowth of bone into immunomodulatory implant 20. In an exemplary embodiment, and as previously described, surface 32 is coated or gradually integrated (e.g., via 3D printing) with a calcium containing compound, and spray 68 is applied to such calcium compound outer portion or coating. Alternatively, surface 32 could be coated with a paste or putty of xenograft or allograft bone containing an antigen and/or a cytokine.

The layered arrangement of immunomodulatory implant 20, for example, a first portion 36A which increases in porosity to its outer surface 32, a calcium compound second portion 36B arranged on the first portion 36A, and an antigen and/or a cytokine carrier third portion arranged on the second portion, ensures that bone grows firmly and reliably into the interstices of immunomodulatory implant 20 and thus improves surgical implant outcomes in skeletal applications. In particular, the application of the antigen and/or cytokine carrier portion activates the innate immune system leading to bone morphogenesis and provides the reason for osteoblastic cells to enter the pores of the immunomodulatory implant 20. In an exemplary embodiment, first portion 36A comprises a metal, for example, titanium, steel, or cobalt-chrome. In an exemplary embodiment, first portion 36A comprises a polymer or plastic, for example, poly ether ether ketone (PEEK).

One of the critical cellular components of bone morphogenesis is the macrophage, also a key player in phagocytosing and eradicating necrotic debris and invading organisms such as bacteria. Macrophages exist in blood as monocytes or M0 cells and circulate through virtually every body organ. M0 macrophages can be activated by stimuli such as trauma debris or bacterial antigens, and follow the chemical gradients provided by the stimuli to the source of either aseptic or septic trauma. Thus, the antigen and/or cytokine and carrier portion attracts M0 macrophages to the cite of immunomodulatory implant 20.

Necrotic debris and antigens, in addition to attracting M0 macrophages, also have the capacity to convert M0 macrophages to M1 macrophages, which are integral in removing necrotic cellular debris and invading organisms such that the stage is set for successful bone repair to begin. M1 or inflammatory or classically activated macrophages aggressively phagocytize debris and alien organisms until none are detected, and then the M1 macrophages transform into M2 or alternatively activated or reparative macrophages, whereupon bone morphogenic repair can begin. Thus, after M1 macrophages phagocytize the antigen and/or encounter the cytokine in the carrier, they convert to M2 macrophages.

M2 macrophages, via released cytokines (in addition to implant carrier cytokines), have the ability to attract mesenchymal stem cells and coordinate their transformation into osteoblasts, which in turn take over the bone formation requirements. M2 macrophages also coordinate the timely release of cytokines like vascular endothelial growth factor (VEGF) to bring necessary nutrients to the injury site by way of nascent vessel formation. Similarly, osteoclasts are transformed by released cytokines and activated to aid in bone remodeling as healing occurs.

It should be appreciated that, in an exemplary embodiment, the implant includes a cytokine alternative or in addition to the antigen component for stimulating/converting the M1 macrophages to M2 macrophages when the M1 macrophages encounter the cytokine. For example, the implant may comprise an antigen/cytokine complex, which is the matrix of the implant that contains some arrangement of antigen and cytokine to activate macrophages.

While surgical insertion of an implant creates traumatic (necrotic) particles through the use of drills and electrocautery, it does not create sufficient debris or a bone implant microenvironment that favors osteogenesis or bony ingrowth. Thus, immunomodulatory implant 20 encourages macrophages to enter its porous surface 32 (if uncoated with a calcium compound) and/or layer 34 (if coated with a calcium compound) through the application of the (bacterial) antigen and/or cytokine and carrier layer, thus resulting in an increased chance that mesenchymal stem cells enter and be suitably transformed into osteoblasts within the confines of the outer perimeter of the implant itself. The macrophage response created by immunomodulatory implant is far more dramatic, and results in better bony fusion, than that of an untreated implant or a collagen sponge carrying growth factors (bone morphogenic proteins), since the antigen and/or cytokine carrier portion of immunomodulatory implant 20 attracts M0 macrophages to the site and quickly transforms them into M1 macrophages.

In an exemplary embodiment, implant 20 comprising entirely autograft, allograft, or xenograft may be treated with a spray or paste containing an antigen and/or cytokine.

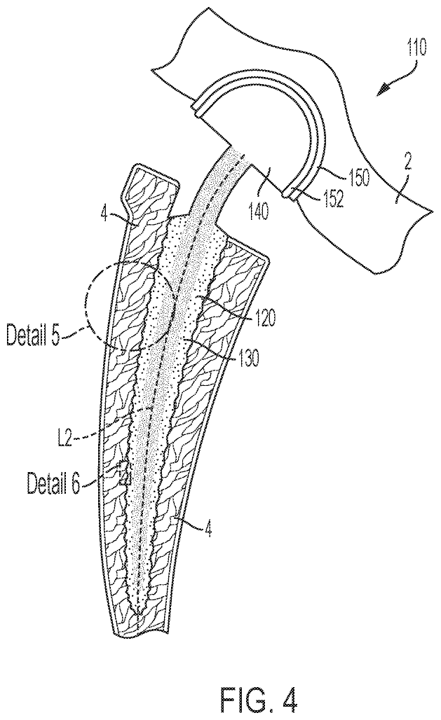

FIG. 4 is a cross-sectional view of immunomodulatory implant system 110 including immunomodulatory implant 120. In FIG. 4, immunomodulatory implant 120 is embodied as a hip replacement implant shown in anatomic position, namely, within femur bone 4 and pelvic bone 2; although, it should be appreciated that immunomodulatory implant 120 can be embodied as any bone implant and the present disclosure is not to be limited to hip replacement implants.

In an exemplary embodiment, and as shown, immunomodulatory implant 120 comprises femoral stem 130 and femoral head 140. Femoral stem 130 is operatively arranged to engage femur bone 4. For example, femoral stem 130 may be arranged in a longitudinal hole arranged in femur bone 4. Femur bone 4 comprises outer cortical bone 4A and inner trabecular or cancellous bone 4B. In an exemplary embodiment, femoral stem 130 is operatively arranged to engage cancellous bone 4B. Femoral head 140 is arranged to pivotably engage bone 2, in particular, the acetabulum of bone 2. In an exemplary embodiment, immunomodulatory implant 120 further comprises acetabular cup 150 arranged to be secured within bone 2. In such exemplary embodiment, femoral head 140 is arranged to pivotably engage acetabular cup 150. In an exemplary embodiment, immunomodulatory implant 120 further comprises liner 152 arranged radially between acetabular cup 150 and femoral head 140. In an exemplary embodiment, liner 152 comprises a polymer.

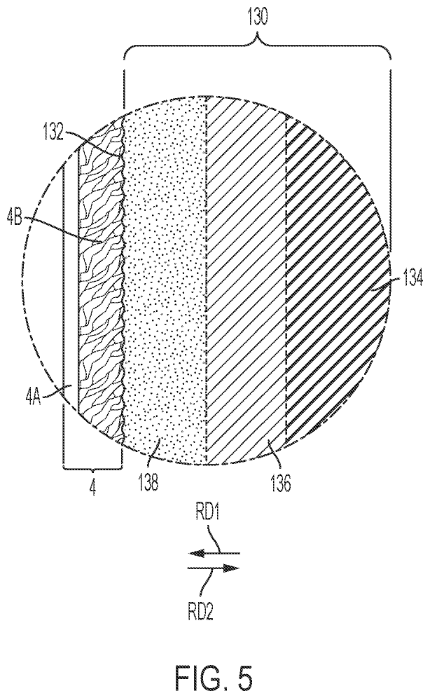

FIG. 5 is a detail view of immunomodulatory implant 120 taken generally along Detail 5 in FIG. 4. Immunomodulatory implant 120 comprises a plurality of portions or layers. For example, immunomodulatory implant 120 comprises portion 134, portion 136, and portion 138. In an exemplary embodiment, portion 138 forms outer surface 132 of immunomodulatory implant 120, which is operatively arranged to engage bone 4. It should be appreciated that outer surface 132 denotes the outer surface of femoral stem 130 and femoral head 140. In an exemplary embodiment, outer surface 132 further denotes the outer surface of acetabular cup 150.

Portion 134 is the inner most portion of immunomodulatory implant 120. In an exemplary embodiment, portion 134 comprises a material, for example a metal such as titanium, steel, or cobalt-chrome, or a polymer or plastic such as PEEK, that is solid or has a low porosity (e.g., less than or equal to 20%). In an exemplary embodiment, the porosity of portion 134 increases from approximately 0% at line L2 radially outward in radial direction RD1 to approximately 20%. In an exemplary embodiment, portion 134 comprises titanium.

Portion 136 is arranged radially outward of, or on, portion 134. In an exemplary embodiment, portion 136 completely encloses portion 134. In an exemplary embodiment, portion 136 comprises a material, for example a metal or a polymer, that has a medium to high porosity (e.g., approximately 20-60%). In an exemplary embodiment, the porosity of portion 136 increases from approximately 20% at its interface with portion 134 radially outward in radial direction RD1 to approximately 50-60%. In an exemplary embodiment, portion 136 comprises titanium.

Portion 138 is arranged radially outward of, or on, portion 136. In an exemplary embodiment, portion 138 completely encloses portion 136. In an exemplary embodiment, portion 138 is a transition layer including both a calcium compound (e.g., calcium carbonate, hydroxyapatite, etc.) and the material of portion 136 (i.e., a metal or polymer). For example, portion 138 is used to transition immunomodulatory implant 120 from a completely metal (or polymer) implant as found in portions 134 and 136 to an at least partial calcium compound implant. This arrangement can be created in a number of ways, for example, 3D printing, spraying a calcium compound on a highly porous outer metal surface of portion 138, adding holes to the outer metal surface of portion 138 (e.g., via a drill bit, shot peening, sandblasting, etc.). In an exemplary embodiment, portion 138 can be formed by adding a paste or a putty to the outer surface of portion 136. The past or putty may be derived from xenograft or allograft bone and include an antigen and/or a cytokine.

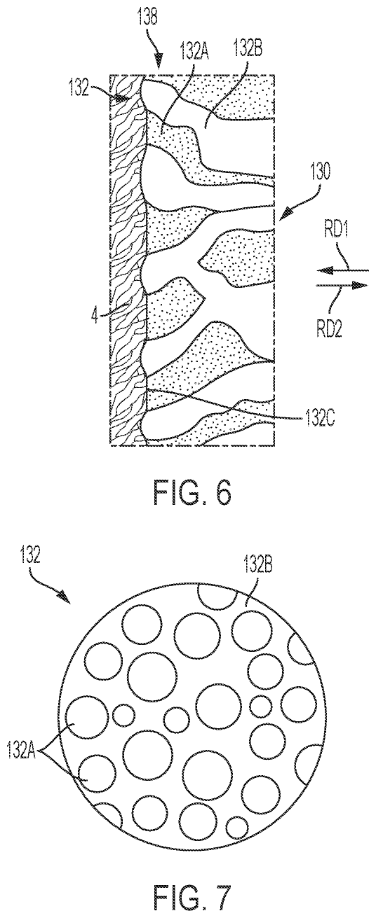

FIG. 6 is a detail view of immunomodulatory implant 120 taken generally along Detail 6 in FIG. 4. In particular, FIG. 6 shows the outermost part of portion 138 which forms outer surface 132 and engages bone 4. Portion 138 comprises first material 132A and second material 132B, both of which form outer surface 132. In an exemplary embodiment, first material 132A is a calcium compound second material 132B is a metal. In an exemplary embodiment, third material 132C may be applied as an outer coating. Third material 132C may comprise an antigen and/or a cytokine. In an exemplary embodiment, the calcium compound material 132A is impregnated or comprises an antigen and/or a cytokine. In such exemplary embodiment, the antigen and/or cytokine may or may not be sprayed as an outer coating.

FIG. 7 is a partial elevational view of an exemplary embodiment of immunomodulatory implant 120. As shown, outer surface 132 of immunomodulatory implant 120 includes metal material 132B (e.g., titanium) enclosing calcium compound material 132A islands. In an exemplary embodiment, calcium compound material 132A islands comprise an antigen and/or a cytokine, for example applied to the surface thereof or 3D printed into the outermost layer thereof. The arrangement of an antigen and/or a cytokine in the calcium compound ensures that bone grows into the holes within metal material 132B after the antigen containing calcium compound is phagocytized (i.e., by M1 monocytes) or the cytokine containing calcium is encountered (i.e., by M1 monocytes). In an exemplary embodiment, the antigen and/or cytokine is applied only to the outer surface of calcium compound material 132A and not to the outer surface of titanium material 132B. In an exemplary embodiment, the outer surface of both calcium compound material 132A and titanium material 132B is coated with the antigen and/or cytokine. In an exemplary embodiment, calcium compound material 132A is impregnated with an antigen and/or a cytokine.

It will be appreciated that various aspects of the disclosure above and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

### REFERENCE NUMERALS

2 Bone or pelvic bone
4 Femoral bone
4A Cortical bone
4B Trabecular or cancellous bone
10 Immunomodulatory implant system
20 Immunomodulatory implant
30 Femoral stem
32 Surface
34 Layer
36A Portion or layer
36B Portion or layer
36C Portion or layer
40 Femoral head
50 Acetabular cup
60 Spraying device
62 Compartment
64 Compartment
66 Nozzle
68 Spray
110 Immunomodulatory implant system
120 Immunomodulatory implant
130 Femoral stem
132 Surface
132A Material
132B Material
132C Material
134 Layer
136 Layer
138 Layer
140 Femoral head
150 Acetabular cup
152 Liner or insert
L1 Line
L2 Line
RD1 Radial direction
RD2 Radial direction

What is claimed is:

1. An immunomodulatory implant, comprising:
an inner portion including a metal or polymer; and
an outer layer arranged on the inner portion and forming an outer surface, the outer layer including a carrier and an immunomodulatory enhancement.

2. The immunomodulatory implant as recited in claim 1, wherein the carrier comprises fibrin.

3. The immunomodulatory implant as recited in claim 1, wherein the carrier comprises hydrogel.

4. The immunomodulatory implant as recited in claim 1, wherein the carrier comprises an adhesive.

5. The immunomodulatory implant as recited in claim 1, wherein the immunomodulatory enhancement comprises an antigen.

6. The immunomodulatory implant as recited in claim 1, wherein the immunomodulatory enhancement comprises a cytokine.

7. The immunomodulatory implant as recited in claim 1, wherein the outer layer comprises a plurality of pores, and the carrier and the immunomodulatory enhancement extend radially inward from the outer surface into the plurality of pores.

8. The immunomodulatory implant as recited in claim 1, further comprising an intermediate layer arranged radially between the inner portion and the outer layer.

9. The immunomodulatory implant as recited in claim 8, wherein the intermediate layer comprises a calcium compound.

10. The immunomodulatory implant as recited in claim 9, wherein the intermediate layer further comprises an antigen and/or a cytokine.

11. The immunomodulatory implant as recited in claim 1, wherein:
the inner portion comprises a porosity and a radial center; and
the porosity increases in a radially outward direction from the radial center.

12. The immunomodulatory implant as recited in claim 1, wherein the carrier comprises a paste or putty derived from xenograft or allograft bone.

13. An immunomodulatory implant, comprising:
an innermost layer including a first porosity, the innermost layer forming a radial center;
an intermediate layer arranged on the innermost layer, the intermediate layer comprising a second porosity, the second porosity being greater than the first porosity; and
an outer layer arranged on the intermediate layer and forming an outer surface, the outer layer including a calcium compound and an immunomodulatory enhancement.

14. The immunomodulatory implant as recited in claim 13, wherein the first porosity increases in a radially outward direction from the radial center to the outer surface.

15. The immunomodulatory implant as recited in claim 14, wherein the second porosity increases in the radially outward direction.

16. The immunomodulatory implant as recited in claim 13, wherein the innermost layer, the intermediate layer, and the outer layer comprise a metal.

17. The immunomodulatory implant as recited in claim 13, wherein the outer surface comprises a first plurality of portions comprising a metal or a polymer and a second plurality of portions comprising the calcium compound.

18. The immunomodulatory implant as recited in claim 17, wherein the second plurality of portions are arranged as islands with respect to the first plurality of portions.

19. The immunomodulatory implant as recited in claim 17, wherein the immunomodulatory enhancement is arranged only on the second plurality of portions.

20. The immunomodulatory implant as recited in claim 16, wherein the implant transitions from 100% metal or polymer material at the radial center radially outward to 100% calcium compound and immunomodulatory enhancement at the outer surface in a graduated fashion.
